(19) United States
(12) Patent Application Publication
(10) Pub. No.: US 2007/0106316 A1
(43) Pub. Date: May 10, 2007

(54) Dural Knife with Foot Plate

(75) Inventor: Ashraf Samy Youssef, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/539,996

(22) Filed: Oct. 10, 2006

Related U.S. Application Data
(60) Provisional application No. 60/596,649, filed on Oct. 10, 2005.

(51) Int. Cl. A61B 17/32

(52) U.S. Cl. 606/167

(57) ABSTRACT

A dural knife with a protective foot-plate for use in surgical procedures. The dural knife includes a movable foot-plate disposed on an end of the scalpel in proximity to the blade. The foot plate uses a hinge joint to attach to the handle at a region near the attachment of a blade to the handle. The foot plate has a ball and socket locking mechanism. The foot-plate can be rotated 90 degrees outward to apply the blade then locked back in place. The device allows cutting of the dural meningeal layer while retracting it away from underlying neurovascular structures and thus protecting them. The device is generally made of stainless steel with a serrated handle and tapered tip with an attached, removable sharp blade. A small dural incision is made to introduce the foot plate underneath. The dura is elevated and divided between the foot plate and the sharp blade. The new device will allow safer opening of the dura in regular cranial and spinal neurosurgical procedures. In complex cranial surgery, it is extremely beneficial in dealing with dural membranes and ligaments along delicate neurovascular structures.

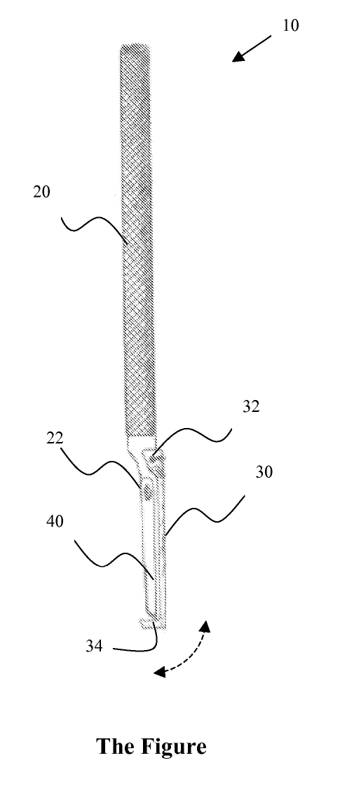

### CROSS REFERENCE TO RELATED APPLICATIONS

This application claims priority to currently pending U.S. Provisional Patent Application 60/596,649, entitled, “Dural Knife with Foot Plate”, filed Oct. 10, 2005, the contents of which are herein incorporated by reference.

### FIELD OF INVENTION

This invention relates to surgical instruments. More particularly this invention relates to a scalpel having a protective foot plate adapted to facilitate the cutting of the dural meningeal layer while protecting the underlying neurovascular structures.

### BACKGROUND OF THE INVENTION

The current techniques of cranial and spinal dural incisions include sharp incisions by scissors and scalpels. Both techniques carry the risk of injury to underlying neurovascular structures by either the sharp blade or the scissors tips. Cranial base surgery may require fine dural incisions along delicate neurovascular structures (for example: cranial nerves and blood vessels). Dividing dural sleeves around cranial nerves may subject them to the risk of injury in the absence of protection from sharp cutting edges. What is needed is a device that allows for the targeted incision of the dura while simultaneously affording protection to the underlying neurovascular structures.

### SUMMARY OF INVENTION

A dural knife with a protective foot-plate for use in surgical procedures. The dural knife includes a movable foot-plate disposed on an end of the scalpel in proximity to the blade. The foot plate uses a hinge joint to attach to the handle at a region near the attachment of a blade to the handle. The foot plate has a ball and socket locking mechanism. The foot-plate can be rotated 90 degrees outward to apply the blade then locked back in place. The device allows cutting of the dural meningeal layer while retracting it away from underlying neurovascular structures and thus protecting them. The device is generally made of stainless steel with a serrated handle and tapered tip with an attached, removable sharp blade. A small dural incision is made to introduce the foot plate underneath. The dura is elevated and divided between the foot plate and the sharp blade. The new device will allow safer opening of the dura in regular cranial and spinal neurosurgical procedures. In complex cranial surgery, it is extremely beneficial in dealing with dural membranes and ligaments along delicate neurovascular structures.

In accordance with the present invention there is provided an improved surgical scalpel. The surgical scalpel includes a body, the body having a first end and a second end, the first end adapted to be grasped by the hand of a user and the second end adapted to securely engage a surgical cutting blade. The surgical scalpel also includes a surgical cutting blade attachment projection for receiving the surgical cutting blade thereon, the cutting blade attachment projection being integrally formed on the second end of the body of the scalpel and extending forwardly therefrom, so that the cutting blade thereon extends forwardly therefrom. The surgical scalpel further includes a protective foot-plate hingedly attached to the second end of the body, the protective foot plate extending substantially coextensively with the surgical cutting blade and in proximity to the blunt aspect of the surgical cutting blade in a first position, the protective foot-plate terminating in a bend extending over the tip of the surgical cutting blade to protect tissues from the tip of the blade during operation, the foot plate extending substantially perpendicularly from the blunt aspect of the surgical blade in a second position, wherein the second position is defined by the limit of hinged rotation of the protective foot-plate away from said first position.

In certain embodiments the surgical scalpel includes a locking mechanism to reversibly fix the protective foot plate in the second position. The locking mechanism can be a ball and socket locking mechanism. In certain embodiments the surgical scalpel includes a textured body to facilitate grasping the scalpel by the hand of a user.

### BRIEF DESCRIPTION OF THE DRAWINGS

For a fuller understanding of the invention, reference should be made to the following detailed description, taken in connection with the accompanying drawings, in which:

The FIGURE is an illustration of a dural knife with a protective foot plate.

### DETAILED DESCRIPTION OF THE PREFERRED EMBODIMENT

The disclosed invention is a dural knife having a protective foot plate. The protective foot plate facilitates the cutting of the dural meningeal layer while protecting the underlying neurovascular structures. The invention will be further described by way of the following non-limiting example.

The dural knife is a surgical device supplied with a protective foot-plate. The portion of the device having the blade and protective-foot plate can be placed through a small incision between the dural layer and underlying neurovascular structures. The dura can then be divided between the sharp edge of the blade and the foot plate. The device can replace the current techniques of dural opening for both cranial and spinal neurosurgical procedures. It will add safety to the dural opening in regular neurosurgical procedures. In complex cranial surgery, it will prove extremely beneficial in dealing with dural membranes and ligaments along delicate neurovascular structures. The device will greatly improve the safety and efficiency of neurosurgical procedures.

Referring to The Figure there is shown an embodiment of the dural knife with protective foot plate 10 according to the present invention. The disclosed embodiment has a handle 20 with a serrated surface over most of the handle to facilitate gripping. The tip of the handle portion tapers at its distal end with a projection 22 adapted to accept the notch of a removable blade 40. Proximal to the site for attaching the blade there is the attachment 32 for the protective foot-plate. The protective footplate attaches to the scalpel using a hinge. The hinge allows for 90 degree movement of the footplate whereby in a first position the foot-plate extends from the tip of the handle and substantially coextensively with a removable blade. From this position the protective foot can be extended away from the blade by employing 90 degree rotation at the hinge, as indicated by the double-headed arrow in The FIGURE, thus arriving at a position forming a right angle in relation to the blade. The protective foot plate further includes a ball and socket locking mechanism to secure the foot plate when rotated away from the blade (not shown). At an end of the foot plate opposite the hinge and proximal to the tip of the removable surgical blade, the terminus 34 of the foot-plate makes a 90 degree bend around the tip of the surgical blade. The terminal bend protects tissues from the tip of the surgical blade when the foot-plate is fixed in the position where the foot-plate extends parallel to the blade. The dural knife will generally be made of stainless steel or other suitable material.

The disclosure of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A surgical scalpel comprising:
a body, the body having a first end and a second end, the first end adapted to be grasped by the hand of a user and the second end adapted to securely engage a surgical cutting blade;
a surgical cutting blade attachment projection for receiving the surgical cutting blade thereon, the cutting blade attachment projection being integrally formed on the second end of the body of the scalpel and extending forwardly therefrom, so that the cutting blade thereon extends forwardly therefrom; and
a protective foot-plate hingedly attached to the second end of the body, the protective foot plate extending substantially coextensively with the surgical cutting blade and in proximity to the blunt aspect of the surgical cutting blade in a first position, the protective foot-plate terminating in a bend extending over the tip of the surgical cutting blade to protect tissues from the tip of the blade during operation, the foot plate extending substantially perpendicularly from the blunt aspect of the surgical blade in a second position, wherein the second position is defined by the limit of hinged rotation of the protective foot-plate away from said first position.

2. The surgical scalpel according to claim 1 further comprising a locking mechanism to reversibly fix the protective foot plate in the second position.

3. The surgical scalpel according to claim 2 wherein the locking mechanism is a ball and socket locking mechanism.

4. The surgical scalpel according to claim 1 further comprising a textured body to facilitate grasping the scalpel by the hand of a user.

5. A surgical scalpel comprising:
a body, the body having a first end and a second end, the first end adapted to be grasped by the hand of a user and the second end adapted to securely engage a surgical cutting blade;
a surgical cutting blade attachment projection for receiving the surgical cutting blade thereon, the cutting blade attachment projection being integrally formed on the second end of the body of the scalpel and extending forwardly therefrom, so that the cutting blade thereon extends forwardly therefrom; and
a protective foot-plate hingedly attached to the second end of the body, the protective foot plate extending substantially coextensively with the surgical cutting blade and in proximity to the blunt aspect of the surgical cutting blade in a first position, the foot plate extending substantially perpendicularly from the blunt aspect of the surgical blade in a second position, wherein the second position is defined by the limit of hinged rotation of the protective foot-plate away from said first position, the protective foot plate terminating in a substantially 90 degree bend at the limit of the extent of the foot plate, whereby the bend covers the tip of the surgical blade in the first position thus protecting tissue from the tip of the blade.

6. The surgical scalpel according to claim 5 further comprising a locking mechanism to reversibly fix the protective foot plate in the second position.

7. The surgical scalpel according to claim 6 wherein the locking mechanism is a ball and socket locking mechanism.

8. The surgical scalpel according to claim 5 further comprising a textured body to facilitate grasping the scalpel by the hand of a user.

9. A surgical scalpel comprising:
a body, the body having a first end and a second end, the first end adapted to be grasped by the hand of a user and the second end adapted to securely engage a surgical cutting blade;
a surgical cutting blade attachment projection for receiving the surgical cutting blade thereon, the cutting blade attachment projection being integrally formed on the second end of the body of the scalpel and extending forwardly therefrom, so that the cutting blade thereon extends forwardly therefrom; and
a protective foot-plate hingedly attached to the second end of the body, the protective foot plate extending substantially coextensively with the surgical cutting blade and in proximity to the blunt aspect of the surgical cutting blade in a first position, the foot plate extending substantially perpendicularly from the blunt aspect of the surgical blade in a second position, wherein the second position is defined by the limit of hinged rotation of the protective foot-plate away from said first position.

10. The surgical scalpel according to claim 9 further comprising a locking mechanism to reversibly fix the protective foot plate in the second position.

11. The surgical scalpel according to claim 10 wherein the locking mechanism is a ball and socket locking mechanism.

12. The surgical scalpel according to claim 9 further comprising a textured body to facilitate grasping the scalpel by the hand of a user.
